(19) United States
(12) Patent Application Publication
(10) Pub. No.: US 2023/0363075 A1
(43) Pub. Date: Nov. 9, 2023

(54) PLASMA TREATMENT DEVICE

(71) Applicant: Clean Crop Technologies, Inc., Holyoke, MA (US)

(72) Inventors: Yaqoot Shaharyar, Northampton, MA (US); Shardul Sreekumar, Easthampton, MA (US); Sandon Hess, Norwalk, CT (US); Rachael Useted, Belchertown, MA (US); Daniel White, Chesterfield, MA (US); Daniel Cavanaugh, Hadley, MA (US)

(21) Appl. No.: 17/751,165

(22) Filed: May 23, 2022

Related U.S. Application Data
(60) Provisional application No. 63/338,295, filed on May 4, 2022.

(51) Int. Cl. H05H 1/24; A61L 2/14

(52) U.S. Cl. CPC H05H 1/2437; A61L 2/14; H05H 2245/40; H05H 2245/36; A61L 2202/11

(57) ABSTRACT

A plasma treatment device includes dielectric plates arranged in parallel, sets of active electrodes disposed on outwardly facing sides of the dielectric plates, respectively, and ground electrodes interposed between inwardly facing sides of the dielectric plates. The sets of active electrodes and the ground electrodes are arranged to define a plasma treatment zone, which exhibits multi-axis symmetry and which is receptive of particles, and are operable to generate plasma for plasma treating the particles therein.

### CROSS-REFERENCE TO RELATED APPLICATIONS

This application is based on and claims priority to U.S. Provisional Patent Application No. 63/338,295, filed on May 4, 2022, and all the benefits accruing therefrom under 35 U.S.C. § 119, the contents of which are incorporated by reference herein in their entirety.

### BACKGROUND

The following description relates to a high-voltage atmospheric pressure dielectric barrier discharge (DBD) reactor that can provide a plasma dose delivery in an infield treatment for disinfection and surface modification of light and powdery substrates.

Substrates with relatively small masses, such as organic powders, e.g., spices, organic or inorganic materials or biologically active materials, such as seeds, can benefit from plasma treatment. Cold plasma treatment in particular is an effective way to disinfect and functionalize the surfaces of these substrates. Indeed, the influence of a cold plasma treatment of various types of seeds can increase the surface area of the seeds and increase the water uptake capacities thereof. Moreover, the action of a plasma on seeds can expose the seeds to reactive species, such as O₃, H₂O₂, NO₂, or NO₃, which can increase the content of malondialdehyde (MDA), a product of lipid oxidation, as well as functionalize the seed coat. This can influence seed permeability. This in turn has the result of improving germination rate, and increased biomass (root length and shoot length), among other advantages compared to untreated seeds).

High-voltage atmospheric pressure plasma (>25 kV peak or 10 kV/mm −500 kV/mm voltage at 1 atm) can be a particularly industrially relevant technology for such substrate/seed treatments. The application of high voltages presents multiple advantages including, but not limited to, a higher power density for a given capacitive load and increased electron density, which decreases the residence time for a given treatment. Moreover, higher voltages are preferred for infield treatments which are important for direct surface functionalization (like seed scarification) because higher voltages allow for increasing the discharge gap. This allows a more commercially viable treatment zone for a wide range of substrates as well as the ability to modulate discharge parameters, like streamer density as a function of the discharge gap. Similarly, for surface discharge reactor systems, higher voltages can result in more electrohydrodynamic thrust as well as a higher concentration of reactive species. Reduced residence times also allow for relatively efficient decoupling between plasma induced surface functionalization and exposure to reactive oxygen species (ROS) and reactive nitrogen species (RNS) with longer half-lives like O₃, thus providing better process control. Enhanced voltages also unlock other useful chemical pathways which are otherwise unavailable at lower voltages. For example, in air, increasing voltage increases a rate of the production of NOR, species which can be a beneficial disinfectant as well as a source of nitrogen for seeds. Nonetheless, there remains a need for improved dielectric barrier discharge reactor configurations for lightweight substrates.

### BRIEF DESCRIPTION

According to an aspect of the disclosure, a plasma treatment device is provided and includes dielectric plates, sets of active electrodes disposed on outwardly facing sides of the dielectric plates, respectively, and ground electrodes interposed between inwardly facing sides of the dielectric plates. The sets of active electrodes and the ground electrodes are arranged to define a plasma treatment zone, which exhibits multi-axis symmetry and which is receptive of particles, and are operable to generate plasma for plasma treating the particles therein.

In accordance with additional or alternative embodiments, the particles include at least one of seeds, spices and inorganic or organic powder particles and the sets of active electrodes are operable at 10-500 kV/cm and with power densities ranging from 0.1-10 W/cm².

In accordance with additional or alternative embodiments, the plasma treatment zone is polygonal.

In accordance with additional or alternative embodiments, the plasma treatment zone is rectangular.

In accordance with additional or alternative embodiments, the plasma treatment zone is elliptical.

In accordance with additional or alternative embodiments, sidewalls of the active electrodes of the sets of active electrodes are recessed inwardly from corresponding sidewalls of the ground electrodes.

In accordance with additional or alternative embodiments, sidewalls of the active electrodes of the sets of active electrodes are flush with corresponding sidewalls of the ground electrodes.

In accordance with additional or alternative embodiments, sidewalls of the active electrodes of the sets of active electrodes overlap with corresponding sidewalls of the ground electrodes.

In accordance with additional or alternative embodiments, a ground electrode strip bisects the plasma treatment zone.

In accordance with additional or alternative embodiments, the dielectric plates are separable for receiving the particles in the plasma treatment zone.

According to an aspect of the disclosure, a plasma treatment method is provided and includes providing a plasma treatment device, loading particles in the plasma treatment zone of the plasma treatment device and applying a voltage between sets of active electrodes and ground electrodes to generate plasma for plasma treating the particles in the plasma treatment zone.

According to an aspect of the disclosure, a plasma treatment device is provided and includes dielectric plates, sets of active electrodes disposed on outwardly facing sides of the dielectric plates, respectively, and ground electrodes interposed between inwardly facing sides of the dielectric plates. The sets of active electrodes and the ground electrodes are arranged to define multiple plasma treatment zones, each of which exhibits multi-axis symmetry and each of which is receptive of particles, and are operable to generate plasma for plasma treating the particles therein.

In accordance with additional or alternative embodiments, the particles include at least one of seeds, spices and inorganic or organic powder particles and the sets of active electrodes are operable at 10-500 kV/cm and with power densities ranging from 0.1-10 W/cm².

In accordance with additional or alternative embodiments, the ground electrodes between neighboring ones of the multiple plasma treatment zones are shared between the neighboring ones of the multiple plasma treatment zones.

In accordance with additional or alternative embodiments, the plasma treatment device is arranged in a stack with additional plasma treatment devices and sets of active electrodes between sequential dielectric plates in the stack are shared between the sequential dielectric plates in the stack.

According to an aspect of the disclosure, a plasma treatment method is provided and includes providing a plasma treatment device, loading particles in plasma treatment zones of the plasma treatment device and applying a voltage between sets of active electrodes and ground electrodes to generate plasma for plasma treating the particles in the plasma treatment zones.

According to an aspect of the disclosure, a plasma treatment device is provided and includes first and second dielectric plates arranged in parallel, a first set of active electrodes disposed in parallel about a first axis on an outwardly facing side of the first dielectric plate, a second set of active electrodes disposed in parallel about the first axis on an outwardly facing side of the second dielectric plate and ground electrodes disposed in parallel about a second axis, which is perpendicular to the first axis, and interposed between inwardly facing sides of the first and second dielectric plates. The first and second sets of active electrodes and the ground electrodes define a plasma treatment zone, which is receptive of particles, and are operable to generate plasma for plasma treating the particles therein.

In accordance with additional or alternative embodiments, the first and second sets of active electrodes and the ground electrodes define multiple plasma treatment zones and ground electrodes between neighboring ones of the multiple plasma treatment zones are shared between the neighboring ones of the multiple plasma treatment zones.

In accordance with additional or alternative embodiments, the plasma treatment device is arranged in a stack with additional plasma treatment devices and first and second sets of active electrodes between sequential dielectric plates in the stack are shared between the sequential dielectric plates in the stack.

According to an aspect of the disclosure, a plasma treatment method is provided and includes providing a plasma treatment device, loading particles in a plasma treatment zone of the plasma treatment device and applying a voltage between sets of active electrodes and the ground electrodes to generate plasma for plasma treating the particles in the plasma treatment zone.

These and other advantages and features will become more apparent from the following description taken in conjunction with the drawings.

### BRIEF DESCRIPTION OF THE DRAWINGS

The subject matter, which is regarded as the disclosure, is particularly pointed out and distinctly claimed in the claims at the conclusion of the specification. The foregoing and other features and advantages of the disclosure are apparent from the following detailed description taken in conjunction with the accompanying drawings in which:

FIG. 1 is a top-down schematic side view of a plasma treatment device in accordance with embodiments;

FIG. 2 is a side schematic side view of the plasma treatment device of FIG. 1 in accordance with embodiments;

FIG. 3 is a side schematic side view of the plasma treatment device of FIG. 1 orthogonal to FIG. 2 in accordance with embodiments;

FIG. 4 is a top-down schematic side view of a plasma treatment device with an elliptical plasma treatment zone in accordance with embodiments;

FIG. 5 is a side schematic side view of the plasma treatment device of FIG. 1 with flush electrodes in accordance with embodiments;

FIG. 6 is a side schematic side view of the plasma treatment device of FIG. 1 with flush electrodes orthogonal to FIG. 5 in accordance with embodiments;

FIG. 7 is a side schematic side view of the plasma treatment device of FIG. 1 with overlapped electrodes in accordance with embodiments;

FIG. 8 is a side schematic side view of the plasma treatment device of FIG. 1 with overlapped electrodes orthogonal to FIG. 7 in accordance with embodiments;

FIG. 9 is a top-down schematic side view of a plasma treatment device with multiple plasma treatment zones in accordance with embodiments; and

FIG. 10 is a side schematic side view of a stack of plasma treatment devices in accordance with embodiments.

These and other advantages and features will become more apparent from the following description taken in conjunction with the drawings.

### DETAILED DESCRIPTION

Although high-voltage plasma treatments can present certain advantages, the high-voltage regime can present significant challenges due to electrohydrodynamic forces, also called ‘ionic wind,’ generated during plasma treatment. Particularly in the case of lighter and smaller substrates, the ionic winds can cause substrate displacement during treatment processes and can push at least a portion of the substrate out of the plasma field. This substrate transport can result in an irregular or ineffective treatment.

Thus, as will be described below, a high-voltage (HV) dielectric barrier discharge (DBD) plasma reactor is provided with geometries and process conditions that improve a uniformity of plasma dosage for substrates that would be otherwise adversely affected by the action of plasma-induced electrohydrodynamic forces. The HV DBD plasma reactor can be operated with sufficient reliability between 10 to 500 kilovolts per centimeter (kV/cm) and with power densities ranging from 0.1 to 10 watts per square centimeter (W/cm²). The HV DBD plasma reactor can be operated in air or with any suitable reactant gas chemistry including, but not limited to, N₂, O₂, CO₂, CO, H₂, NH₃, or any combination thereof.

Disclosed is a plasma treatment device that includes dielectric plates arranged in parallel, sets of active electrodes disposed on outwardly facing sides of the dielectric plates and ground electrodes interposed between inwardly facing sides of the dielectric plates. The active electrodes and the ground electrodes are arranged to define a plasma treatment zone. The plasma treatment zone exhibits multi-axis symmetry and is receptive of particles. The active electrodes and the ground electrodes are operable to generate plasma for plasma treating the particles in the plasma treatment zone.

In some cases, overlapping edges of the active (i.e., high voltage) and ground electrodes represent a particular type of surface dielectric barrier discharge reactor. In these or other cases, ionic winds are generated at each site which interact with the substrate causing local turbulence even as symmetrical interactions can reduce a propensity of the substrate to be blown out of the reaction zone.

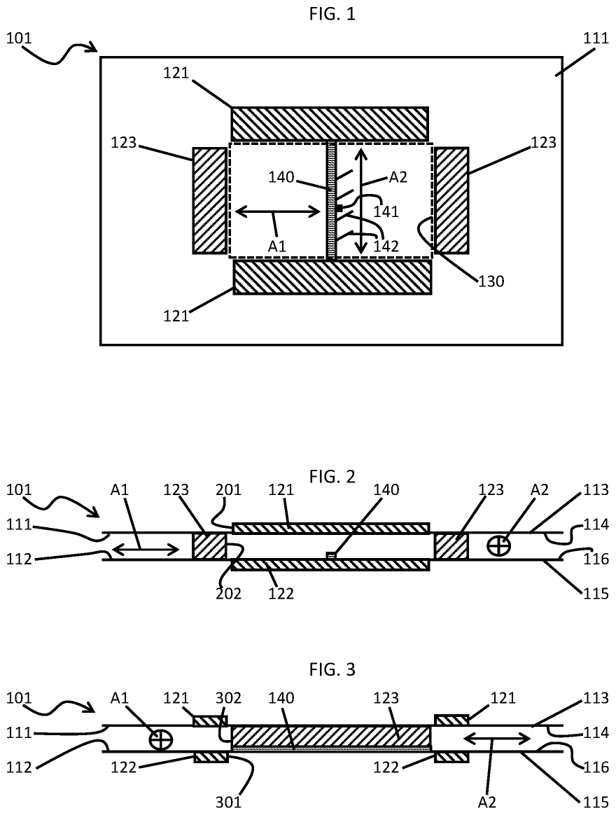

With reference to FIGS. 1-3, a plasma treatment device 101 is provided and includes a first dielectric plate 111 and a second dielectric plate 112. The first and second dielectric plates 111 and 112 are arranged in parallel with one another. The first dielectric plate 111 has an outwardly facing side 113, which faces away from the second dielectric plate 112, and an inwardly facing side 114, which faces toward the second dielectric plate 112. The second dielectric plate 112 has an outwardly facing side 115, which faces away from the first dielectric plate 111, and an inwardly facing side 116, which faces toward the first dielectric plate 111. The plasma treatment device 101 further includes a first set of active electrodes 121, a second set of active electrodes 122 and ground electrodes 123. Each active electrode of the first set of active electrodes 121 is disposed on the outwardly facing side 113 of the first dielectric plate 111. Each active electrode of the second set of active electrodes 122 is disposed on the outwardly facing side 115 of the second dielectric plate 112. Each of the ground electrodes 123 is interposed between the inwardly facing sides 114 and 116 of the first and second dielectric plates 111 and 112, respectively. The first and second sets of active electrodes 121 and 122 and the ground electrodes 123 are arranged to define a plasma treatment zone 130. The plasma treatment zone 130 exhibits multi-axis symmetry about at least first axis A1 and second axis A2 owing to the respective and relative arrangements of the first and second sets of active electrodes 121 and 122 and the ground electrodes 123. The plasma treatment zone 130 is receptive of particles, such as seed or powder particles, and the first and second sets of active electrodes 121 and 122 and the ground electrodes 123 are operable to generate plasma for plasma treating the particles in the plasma treatment zone 130.

In accordance with additional embodiments, the plasma treatment device 101 can further include a ground electrode strip 140. As shown in FIGS. 1 and 3, the ground electrode strip 140 can be disposed to bisect the plasma treatment zone 130. As such, when the first and second sets of active electrodes 121 and 122 and the ground electrodes 123 are operated to generate plasma for plasma treating the particles in the plasma treatment zone 130, the ground electrode strip 140 effectively causes plasma generation to be focused in two sub-zones on opposite sides of the ground electrode strip which in turn allows for two sets of particles to be plasma treated separately within the plasma treatment zone 130 at one time. A cross-section of the ground electrode strip 140 can be any suitable shape, rectilinear or curvilinear, and can be square, rectangular, circular, or elliptical.

In detail, the ground electrode strip 140 causes a volumetric plasma wall to be generated in the middle of the plasma treatment zone 130. Light substrates in particular get pushed toward this volumetric plasma wall due to action of corresponding surface discharge reactor arrangements at corners of the plasma treatment zone 130. The substrates collide with the plasma wall and get pushed back. The continuous interaction between the surface discharge arrangement and the volumetric plasma wall causes a turbulent motion of light substrate particles thereby increasing the contact efficiency between the reactive gas species and the substrate.

To aid in the generation of the turbulent motion, the ground electrode strip 140 can include surface features that tend to increase the turbulence. These surface features can include, but are not limited to, turbulators 141 and/or louvers or baffles 142 whose angle of attack relative to the ground electrode strip 140 can be controllable.

In accordance with additional embodiments, the first and second dielectric plates 111 and 112 can be separable from one another to allow for user access to the plasma treatment zone 130, so that a user can pour or deposit a substrate or the particles in the plasma treatment zone 130, and are re-attachable for plasma treatment operations to commence.

The plasma treatment device 101 is configured and operable as a high-voltage (HV) dielectric barrier discharge (DBD) plasma reactor. This HV DBD plasma reactor is operable at 10 to 500 kV/cm, 20 to 450 kV/cm, 50 to 400 kV/cm, or 100 to 300 kV/cm, and with power densities ranging from 0.1 to 10 W/cm², 0.2 to 8 W/cm², or 1 to 5 W/cm². The DBD may be operated using air, or any other suitable reactant gas, including, but not limited to, N₂, O₂, CO₂, CO, H₂, NH₃ or any combination thereof. The first and second sets of active electrodes 121 and 122 and the ground electrodes 123 can be formed of a metal such as aluminum (Al), copper (Cu), silver (Ag), tungsten (W), titanium (Ti), or an alloy thereof Use of aluminum is mentioned.

The first and second dielectric plates 111 and 112 can be formed of suitable dielectric material, and may be comprise a glass, a ceramic, a polymeric material, or a combination thereof. Suitable glasses include borosilicate glass (e.g., PYREX) or fused silica (e.g., quartz, SiO₂). Representative ceramic materials include alumina (Al₂O₃), silicon carbide (SiC), silicon nitride (SiN), zirconia (ZrO₂), or mica. Representative polymeric materials include polycarbonate, polyethylene (e.g., high-density polyethylene (HDPE) or ultrahigh molecular weight polyethylene (UHMW), polypropylene, a polyimide such as KAPTON, or a polyether ether ketone (PEEK) can also be used as a dielectric material. A thickness of the first and second dielectric plates 111 and 112 can be about 0.05 to 1 cm and can be selected to control a nature and intensity of plasma discharge.

In accordance with embodiments, the plasma treatment zone 130 can be formed with most any regular polygonal shape. For example, as shown in FIG. 1, the plasma treatment zone 130 can be rectangular. In this exemplary case, the active electrodes of the first set of active electrodes 121 are disposed in parallel with one another about the first axis A1 on the outwardly facing side 113 of the first dielectric plate 111, the active electrodes of the second set of active electrodes 122 are disposed in parallel with one another about the first axis A1 on the outwardly facing side 115 of the second dielectric plate 112 and the ground electrodes are disposed in parallel with one another about a second axis A2, which is perpendicular to the first axis A1, and are interposed between the inwardly facing sides 114 and 116 of the first and second dielectric plates 111 and 112. With this construction, the plasma treatment zone 130 with the exemplary rectangular shape is identified in FIG. 1 by the dashed line.

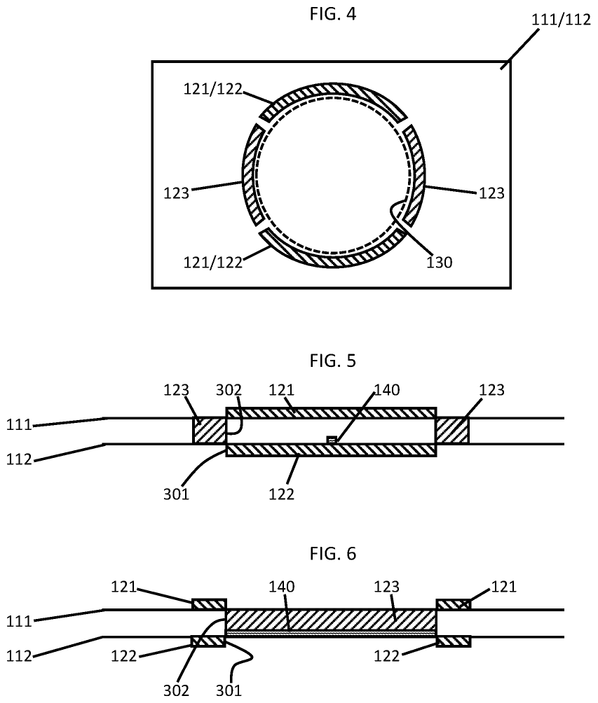

With reference to FIG. 4 and in accordance with embodiments, the plasma treatment zone 130 can be formed with most any regular elliptical shape. For example, as shown in FIG. 4, the plasma treatment zone 130 can be circular. In this exemplary case, the active electrodes of the first set of active electrodes 121 are oppositely arced and disposed in parallel with one another about a first axis A1 on the outwardly facing side 113 of the first dielectric plate 111, the active electrodes of the second set of active electrodes 122 are oppositely arced and disposed in parallel with one another about the first axis A1 on the outwardly facing side 115 of the second dielectric plate 112 and the ground electrodes are oppositely arced and disposed in parallel with one another about a second axis A2, which is perpendicular to the first axis A1, and are interposed between the inwardly facing sides 114 and 116 of the first and second dielectric plates 111 and 112. With this construction, the plasma treatment zone 130 with the exemplary elliptical/circular shape is identified in FIG. 4 by the dashed line.

The following description will relate to the embodiments of the plasma treatment device 101 of FIGS. 1-3 (and FIGS. 5 and 6 and FIGS. 7 and 8, to be described below). This is being done for clarity and brevity and is not intended to otherwise limit the scope of the description or the application as a whole.

As shown in FIG. 2, sidewalls 201 of the active electrodes of the first and second sets of active electrodes 121 and 122 are recessed inwardly along the first axis A1 from corresponding sidewalls 202 of the ground electrodes 123. Similarly, as shown in FIG. 3, sidewalls 301 of the active electrodes of the first and second sets of active electrodes 121 and 122 are recessed inwardly along the second axis A2 from corresponding sidewalls 302 of the ground electrodes 123.

With the arrangement of FIGS. 1-3 described above, ionic winds resulting from plasma discharge caused by the activation of the first and second sets of active electrodes 121 and 122 and the ground electrodes 123 are directed in opposite directions along the first axis A1 and in opposite directions along the second axis A2. The oppositely directed, multi-axial forces tend to prevent leakage of any of the particles out of the plasma treatment zone 130.

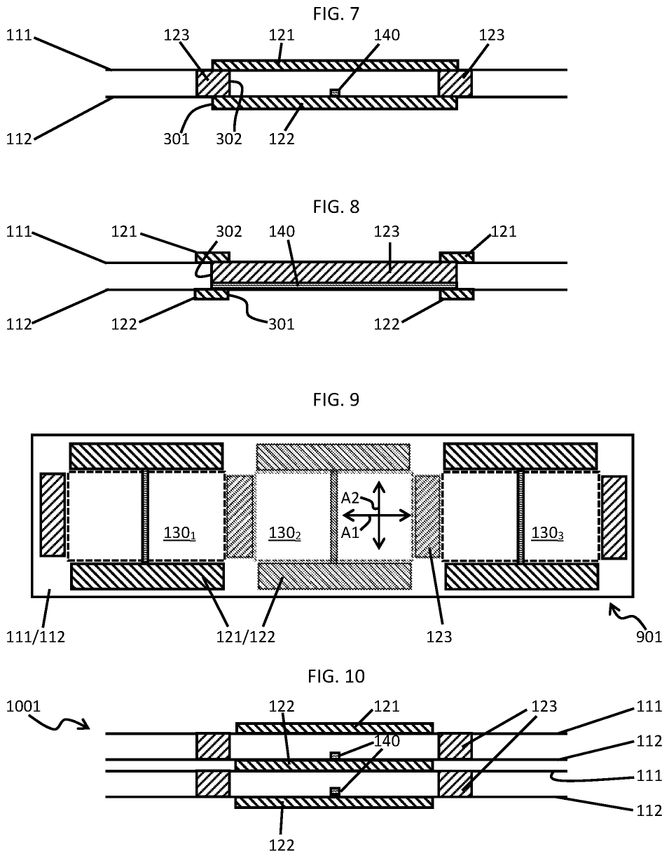

With reference to FIGS. 5 and 6 and FIGS. 7 and 8, alternative embodiments are possible. For example, as shown in FIG. 5, the sidewalls 201 of the active electrodes of the first and second sets of active electrodes 121 and 122 are flush with the corresponding sidewalls 202 of the ground electrodes 123. Similarly, as shown in FIG. 6, the sidewalls 301 of the active electrodes of the first and second sets of active electrodes 121 and 122 are flush with the corresponding sidewalls 302 of the ground electrodes 123. As another example, as shown in FIG. 7, the sidewalls 201 of the active electrodes of the first and second sets of active electrodes 121 and 122 overlap with the corresponding sidewalls 202 of the ground electrodes 123. Similarly, as shown in FIG. 8, the sidewalls 301 of the active electrodes of the first and second sets of active electrodes 121 and 122 overlap with the corresponding sidewalls 302 of the ground electrodes 123. It is to be understood, however, that still other embodiments are possible including combination of those of FIGS. 1-3, FIGS. 5 and 6 and FIGS. 7 and 8. In an case, the ionic winds resulting from the plasma discharge caused by the activation of the first and second sets of active electrodes 121 and 122 and the ground electrodes 123 are directed in opposite directions along the first axis A1 and in opposite directions along the second axis A2. As noted above, the oppositely directed, multi-axial forces tend to prevent leakage of any of the particles out of the plasma treatment zone 130.

With reference to FIG. 9, a plasma treatment device 901 is provided and is similar to plasma treatment device 101 in most respects. As such, like elements have like reference numerals and will not be re-described. As shown in FIG. 9, the plasma treatment device 901 is characterized in that the first and second sets of active electrodes 121 and 122 and the ground electrodes 123 are arranged to define multiple plasma treatment zones 130₁, 130₂ and 130₃. Each of the multiple plasma treatment zones 130₁, 130₂ and 130₃ exhibits multi-axis symmetry about at least the first axis A1 and the second axis A2. Each of the multiple plasma treatment zones 130₁, 130₂ and 130₃ is separately or commonly receptive of the particles, and the first and second sets of active electrodes 121 and 122 and the ground electrodes 123 are operable to generate plasma for plasma treating the particles in each of the multiple plasma treatment zones 130₁, 130₂ and 130₃.

In accordance with embodiments, ground electrodes 123 interposed between neighboring ones of the multiple plasma treatment zones 130₁, 130₂ and 130₃ can be shared between the neighboring ones of the multiple plasma treatment zones 130₁, 130₂ and 130₃.

With reference to FIG. 10, the plasma treatment device 101 of FIGS. 1-8 and/or the plasma treatment device 901 of FIG. 9 can be arranged in a stack 1001 with one or more additional plasma treatment devices 1002 (the embodiment of FIG. 10 is illustrated based on the embodiments of FIGS. 1-8, for clarity and brevity and is not intended to otherwise limit the scope of the description or the application as a whole). In these or other cases, the first and second sets of active electrodes 121 and 122 between sequential dielectric plates 111 and 112 in the stack 1001 are shared between the sequential dielectric plates 111 and 112 in the stack 1001.

Technical effects and benefits of the present disclosure are the provision of reactor systems that ensure that a uniform plasma dose is delivered to light and smaller substrates which would otherwise experience significant movement under the action of plasma-induced electrohydrodynamic forces. Restricting the motion of the substrates (i.e., seeds, spices and inorganic or organic powder, etc.) in plasma and utilization of smaller reactor volumes ensures that uniform exposure is maintained and hence any plasma-related effects like surface modification or disinfection are observed all across the treated substrate volume thereby increasing the value proposition of the process. Additionally, utilization of the above designs enables the use of direct high-voltage treatments that would not have been otherwise possible.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

What is claimed is:

1. A plasma treatment device, comprising:
dielectric plates;
sets of active electrodes disposed on outwardly facing sides of the dielectric plates, respectively; and
ground electrodes interposed between inwardly facing sides of the dielectric plates,
the sets of active electrodes and the ground electrodes being arranged to define a plasma treatment zone, which exhibits multi-axis symmetry and which is receptive of particles, and being operable to generate plasma for plasma treating the particles therein.

2. The plasma treatment device according to claim 1, wherein:
the particles comprise at least one of seeds, spices and inorganic or organic powder particles, and
the sets of active electrodes are operable at 10-500 kV/cm and with power densities ranging from 0.1-10 W/cm².

3. The plasma treatment device according to claim 1, wherein the plasma treatment zone is polygonal.

4. The plasma treatment device according to claim 1, wherein the plasma treatment zone is rectangular.

5. The plasma treatment device according to claim 1, wherein the plasma treatment zone is elliptical.

6. The plasma treatment device according to claim 1, wherein sidewalls of the active electrodes of the sets of active electrodes are recessed inwardly from corresponding sidewalls of the ground electrodes.

7. The plasma treatment device according to claim 1, wherein sidewalls of the active electrodes of the sets of active electrodes are flush with corresponding sidewalls of the ground electrodes.

8. The plasma treatment device according to claim 1, wherein sidewalls of the active electrodes of the sets of active electrodes overlap with corresponding sidewalls of the ground electrodes.

9. The plasma treatment device according to claim 1, further comprising a ground electrode strip bisecting the plasma treatment zone.

10. The plasma treatment device according to claim 1, wherein the dielectric plates are separable for receiving the particles in the plasma treatment zone.

11. A plasma treatment method, comprising:
providing the plasma treatment device of claim 1;
loading the particles in the plasma treatment zone; and
applying a voltage between each of the sets of active electrodes and the ground electrodes to generate the plasma for plasma treating the particles in the plasma treatment zone.

12. A plasma treatment device, comprising:
dielectric plates;
sets of active electrodes disposed on outwardly facing sides of the dielectric plates, respectively; and
ground electrodes interposed between inwardly facing sides of the dielectric plates,
the sets of active electrodes and the ground electrodes being arranged to define multiple plasma treatment zones, each of which exhibits multi-axis symmetry and each of which is receptive of particles, and being operable to generate plasma for plasma treating the particles therein.

13. The plasma treatment device according to claim 12, wherein:
the particles comprise at least one of seeds, spices and inorganic or organic powder particles, and
the sets of active electrodes are operable at 10-500 kV/cm and with power densities ranging from 0.1-10 W/cm².

14. The plasma treatment device according to claim 12, wherein ground electrodes between neighboring ones of the multiple plasma treatment zones are shared between the neighboring ones of the multiple plasma treatment zones.

15. The plasma treatment device according to claim 12, wherein:
the plasma treatment device is arranged in a stack with additional plasma treatment devices, and
sets of active electrodes between sequential dielectric plates in the stack are shared between the sequential dielectric plates in the stack.

16. A plasma treatment method, comprising:
providing the plasma treatment device of claim 12;
loading the particles in the plasma treatment zones; and
applying a voltage between each of the sets of active electrodes and the ground electrodes to generate the plasma for plasma treating the particles in the plasma treatment zones.

17. A plasma treatment device, comprising:
first and second dielectric plates arranged in parallel;
a first set of active electrodes disposed in parallel about a first axis on an outwardly facing side of the first dielectric plate;
a second set of active electrodes disposed in parallel about the first axis on an outwardly facing side of the second dielectric plate; and
ground electrodes disposed in parallel about a second axis, which is perpendicular to the first axis, and interposed between inwardly facing sides of the first and second dielectric plates,
the first and second sets of active electrodes and the ground electrodes defining a plasma treatment zone, which is receptive of particles, and being operable to generate plasma for plasma treating the particles therein.

18. The plasma treatment device according to claim 17, wherein:
the first and second sets of active electrodes and the ground electrodes define multiple plasma treatment zones, and
ground electrodes between neighboring ones of the multiple plasma treatment zones are shared between the neighboring ones of the multiple plasma treatment zones.

19. The plasma treatment device according to claim 18, wherein:
the plasma treatment device is arranged in a stack with additional plasma treatment devices, and
first and second sets of active electrodes between sequential dielectric plates in the stack are shared between the sequential dielectric plates in the stack.

20. A plasma treatment method, comprising:
providing the plasma treatment device of claim 17;
loading the particles in the plasma treatment zone; and
applying a voltage between each of the sets of active electrodes and the ground electrodes to generate the plasma for plasma treating the particles in the plasma treatment zone.
